(19) United States
(12) Patent Application Publication
(10) Pub. No.: US 2023/0223137 A1
(43) Pub. Date: Jul. 13, 2023

(54) Methods and Systems for Operative Analysis and Management

(71) Applicant: Smartjoint Inc., Dover, NH (US)

(72) Inventors: Tyler Vachon, Cardiff-by-the-Sea, CA (US); Matthew Provencher, Edwards, CO (US); Tyler Zajac, San Diego, CA (US)

(21) Appl. No.: 18/095,311

(22) Filed: Jan. 10, 2023

Related U.S. Application Data
(60) Provisional application No. 63/298,109, filed on Jan. 10, 2022.

(51) Int. Cl. G16H 30/40; G06V 20/40; G06V 10/70; G06T 7/00; G16H 20/40

(52) U.S. Cl. CPC G16H 30/40; G06V 20/49; G06V 20/41; G06V 10/70; G06T 7/0014; G16H 20/40; G06V 2201/034; G06T 2207/10016; G06T 2207/30196

(57) ABSTRACT

Embodiments of the application provide methods and devices for analyzing surgeries. It may include recording images of a surgery with a camera, wherein the images may include a visual element chosen from a surgeon's hands during the surgery, a patient's surgery area, equipment used in a surgery, instruments used in a surgery and the like; saving images of a surgery; displaying a timestamp in images; chapterizing the images into different chapters; leveraging additional radiologic imaging clinical data; maximizing treatment cost/benefit; and perhaps even analyzing recorded images of a surgery. Embodiments may use artificial intelligence, computer learning, and machine learning.

This U.S. Non-provisional patent application claiming priority to and the benefit of U.S. Provisional patent application no. 63/298,109 filed Jan. 10, 2022, hereby incorporated by reference herein in its entirety.

### BACKGROUND OF THE INVENTION

There is variability in the performance of surgeons based on initial training, ongoing training and skill decline, possibly related to lack of volume to maintain proficiency. Given the typical individual nature of surgical practice, it can be difficult for surgeons to gauge their performance compared with peers and there is no accurate and reliable method to do this at scale. Outcomes data can show variability however it lacks specificity on which portions of the surgical procedure demonstrate greatest variation from the norm.

There is no scalable, reproducible method to assess and track intra and inter surgeon operative technique variability.

In addition, there is no scalable, reproducible method to assess and track patient outcomes based on clinical information, patient wearable data, and even peri-operative imaging such as MRI, CT scan and X-rays.

Artificial intelligence (“AI”) and its subsets of machine learning and computer vision are gaining traction in the media however the business and clinical use cases have yet to be fully appreciated. Many AI tools on the market have fallen into the unfortunate trap that if something can be done, it should be done. For example, there are 130 FDA 510 k cleared AI algorithms in radiology yet very few have a sustainable business model. The focus was on the technology and not the clinical problem. Learning from this industry pitfall, it is desirable to address the problem of surgeon variability and evaluate the tools available to assist.

In the past, elementary overall outcomes such as operative time or cases per day are the only methods to assess surgeon efficiency. There is no automated system for case timing, instrument identification and use, technique tracking, and the like.

While it may not be feasible for a team of seasoned surgeons to watch every surgeon and create a scorecard by consensus, it is desirable to capture data from surgeries, store it properly, and even identify trends while applying algorithms perhaps trained by seasoned surgeons. This type of scorecard could be useful to assess skill drift after initial and even subsequent training as well as identify divergence from geographic or sub specialty specific best practices. Furthermore, comprehensive longitudinal evaluation of all available patient data could be useful to predict patient outcomes perhaps with enough precision to decrease overall cost in the fully capitated healthcare financial model.

### SUMMARY OF THE INVENTION

The present application includes a variety of aspects, which may be selected in different combinations based upon the particular application or needs to be addressed. In various embodiments, the application may include methods and systems for analyzing and managing surgeries.

It is an object of the application to analyze the case timing of surgical steps. As a non-limiting example, how much time is spent on the diagnostic portion of a procedure versus the fixation/remedy portion of the procedure.

It is another object of the application to analyze the steps in a surgery perhaps with what specific instruments and timeframe thereof. As a non-limiting example, a scope with an arthroscopic probe vs biter, vs guide instrument to do the case - how much/proportion is used.

It is yet another object of the application to analyze the instruments (e.g., tools and equipment, etc.) used during surgery.

Is another object of the application to evaluation the implications for instrumentation use during a surgery, for example, which ones are used most during a case, time on procedure specific portions of case, and what equipment is necessary and/or utilized.

It is yet another object of the application to track evolution of surgery techniques over time perhaps with recognition of time per step, instruments used, and historically compare.

It is an object of the application to provide a surgical scorecard and perhaps even an automated surgical scorecard for surgeries and the like.

It is yet another object of the application to leverage patient collected, as well as patient centered clinical and even radiologic imaging data to maximize clinical outcomes and minimize cost.

Naturally, further objects, goals and embodiments of the application are disclosed throughout other areas of the specification, claims, and drawings.

### BRIEF DESCRIPTION OF THE DRAWINGS

FIG. 1 is a schematic representation of surgery analysis and management in accordance with some embodiments.

FIG. 2 is a schematic representation of pre-surgery analysis in accordance with some embodiments.

FIG. 3 is a schematic representation of surgery analysis and management in accordance with some embodiments.

FIG. 4 is a schematic representation of surgery analysis and management in accordance with some embodiments.

FIG. 5 is a schematic representation of surgery analysis and management in accordance with some embodiments.

FIGS. 6-11 show photographs of images taken during a surgery in accordance with some embodiments.

### DETAILED DESCRIPTION OF THE INVENTIONS

It should be understood that embodiments include a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the application may provide a method to leverage pre and even postoperative clinical and imaging data as well as capture, interpret and display intraoperative milestones to determine and improve intra and inter surgeon variability, operative documentation and patient outcomes. It may also provide a method to capture, interpret and display intraoperative milestones to determine and improve intra (within) and inter (to place) surgeon variability.

Accurate identification of operative instruments and intra-articular structures using computer vision can be systematically applied to track surgical steps, with time stamps, to create dynamic surgeon scorecards and ultimately improve surgical outcomes at scale. Surgeons may be able operate on patients in a typical fashion while data is collected. Once best practices may be identified, feedback can be provided such as but not limited to improving instrument selection, instrument placement, search patterns, instrument use duration, and the like. Surgical leadership can track variability between individuals and groups and with feedback there may be decreased variability and even increased quality in surgeries. Embodiments of the application can be applied to all aspects of surgery, in education and training in the lab environment, in enhancing surgical proficiency and skill, and the like. The nature of data collection can allow a much more thorough and specific evaluation of an individual surgeon, practice, graduate medical education training, post graduate training, skills labs, and the like.

In embodiments, a camera surgical scope device may be inserted in a patient which may be a human or another animal. When a surgical instrument may be used or even inserted in a patient, the instrument may be identified and perhaps even the event may be time stamped. When an anatomic or even pathologic structures are within a field of view a camera, the structure may be identified and the event may be time stamped. Item identification and even associated time stamps may be collected for each surgeon, surgery, and the like. This information may be analyzed, manipulated, and even displayed on a data presentation mechanism perhaps for leadership action, surgeon feedback, and the like.

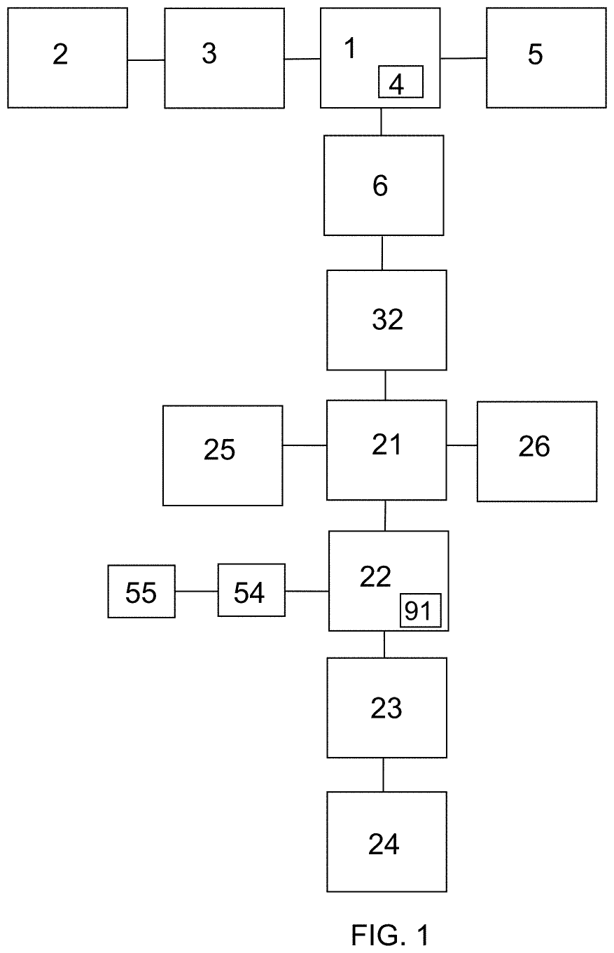

Embodiments of the application may provide a method for analyzing surgeries comprising the steps of recording images (1) of a surgery (2) with a camera (3), wherein images comprise a visual element (4) chosen from a surgeon's hands during a surgery, a patient's surgery area, equipment used in surgery, and instruments used in surgery; saving images of a surgery; displaying a timestamp (5) in the images; chapterizing the images of a surgery into different chapters (6); and perhaps even analyzing (32) recorded images of a surgery as may be understood from FIG. 1. Images may be still images, moving images, such as a video or the like and a camera may be any kind device for capturing a photographic or video images including a video camera. A camera may capture visual elements (4) which may include but is not limited to the surgeon's hands, other's hands, hand placement, the patient's surgery area, equipment used, instruments used, and the like. Images may be saved using a digital memory or the like on a computer memory. In the saved images, a timestamp (5) may be added. This may be the time of the surgery and may even be a running time of the surgery included on the images. The time may be reset at the beginning of the surgery, may even be reset during the surgery perhaps when a new event occurs, may be reset for each chapter, or the like. The surgery images may be divided into chapters perhaps to provide chapterizing of the images. Chapters may be designated by categories such as when specific equipment is used, when specific instrument is used, each surgical step, when a new instrument is used, when an instrument is removed, and the like.

From such data which may be obtained from a plurality of surgeries, institutional or even industry standards may be determined, real time feedback can be provided with multiple comparisons of real-time time stamps to historical data. In analyzing (32) recorded images of a surgery it may include identifying anatomic structures of a patient's surgery area; identifying pathologic structures of a patient's surgery area; identifying each equipment or instrument used in a surgery; identifying equipment or instrument duration of use; identifying equipment or instrument placement (e.g., location of instrument on a structure); identifying equipment or instrument selection; identifying surgeon technique in a surgery; comparing a surgery with recorded images of another surgery; any combination thereof, or the like. Analyzing images from a surgery may also include analysis from many different surgeries and may provide collected data (21). In some embodiments, collected data (21) may be utilized in pre-operation evaluation (26). Collected data (21) may include electronic medical records or electronic health records and the like.

In embodiments, feedback loops may be used perhaps to improve surgical performance. A platform may collect granular data from the recorded images of surgeries perhaps to allow for objective comparison to recognized best practices, display variability, and even track progress. A camera can collect images of the anatomic structures and even surgical instruments and a computing device can identify them and even create an associated time stamp. For each different surgical procedure, data points can be collected and analyzed. By collecting each specific data point during an operation, the surgical steps may be chapterized and analyzed perhaps to determine best practices. Best practices can be shared with surgical teams, training institutions, and individual surgeons perhaps as a process improvement mechanism. Once institutional or industry standards may be determined, real time feedback can be provided during a surgery perhaps with multiple comparisons of real-time time stamps to historical data. In some embodiments, specific data points may be collected from past surgeries and may be used to prompt to a surgeon during surgery. Real-time analysis (54) of a surgery may be conducted during a surgery and it may be based on a comparison of collected data and data input from the currently proceeding surgery. Such analysis may provide a recommendation (55) to a surgeon during the surgery.

By establishing “chapters” for common surgical procedures, a more granular evaluation process of evaluation can be possible. Demarcation between chapters may be determined when surgical instruments are placed or even withdrawn from the field of view of a camera. A training a computer vision platform may then identify and even time stamp images taken from the camera of the surgery and data can be collected at scale. Once data can be accurately and efficiently gathered, individual, practice, regional and other scorecards can be created.

Embodiments may provide the collect and even parsing of large amounts of intra-operative imaging and surgical data. Computer vision, artificial intelligence, or even machine learning algorithms may be utilized with image labeling, testing, and even validation. Such programming may be initiated by machine learning engineers, data scientists, medical domain experts perhaps for anatomic and surgical hardware, and the like.

Through the use of computer vison, artificial intelligence, or even machine learning, algorithmic data and computing may isolate, define, identify anatomic structures, identify pathologic structures (e.g., torn, inflamed, injured structures), and perhaps identify or recommend specific instrumentation, surgical strategies, and the like. An outcome from such computing may include, but is not limited to, case step timing instrument use and timing, instrument location and timing (e.g., how long in particular area of the surgical area), tracking historical data of use and time, comparing historical data, performing benchmark comparisons, and the like. Using historical data, identification of best practices may be maximized and a feedback loop perhaps using a dashboard may be created. A dynamic surgical scorecard (22) may be created perhaps based on collected data (21). A scorecard may provide information including but not limited to camera surgical scope device, surgical instruments, anatomic or pathologic structures, human or another animal, on premises or cloud based computational entity, data presentation mechanism - such as a dashboard, the surgeon, and the like. A scorecard may be created for agenda (91) such as a surgeon, type of surgery, practice of surgeons, type of instrument used, type of instrument used, type of surgery area, and the like.

In embodiments, the application may include: data output; a surgeon/staff interface; data shared with surgeon, vendor, staff, and the like; implementation of time stamps in surgical images/videos for use of instruments; identification of pathology related to instrument use and even tying in the pathologic entities to the instruments and treatment; creating an operative report from data such as what instruments were used are tied with specific CPT codes for coding procedures; creating a report card from data perhaps customized to each surgeon and or user input; and the like.

Other embodiments may include historical controls; regional and even national benchmark comparisons; evolution of techniques from a database perhaps to show old techniques and current techniques. Data and analysis thereof may be used for identification of gaps and even needs for vendors perhaps in terms of what surgeries take longest, need multiple instruments, are slower, and the like perhaps to help with improved development of techniques.

Within the past five years, the accessibility of machine learning algorithms have significantly improved. Whereas previously a team of on staff data scientists and machine learning engineers may have been required to have a functioning platform, now smaller teams can be contracted for specific algorithmic development.

Computer programs including computer vision, artificial intelligence, and machine learning algorithms can be used in various embodiments of the application. As a non-limiting example, implementation started with a single knee structure, anterior cruciate ligament, to assess the cost, in time and money, to create a usable algorithm. The usual steps of accurate labeling data, training and testing the algorithm were completed. The algorithm was tested on four new video samples with about 100% specificity and about 99.8% sensitivity. As such, apart from a few frames of non-recognition, there were zero false positives or negatives. Within the limits of this small sample size, this provides an encouraging result and warrants further evaluation.

Building on this success, expanding to additional knee structures and operative instruments may be desirable to further move toward accurate artificial intelligence enabled tools to track surgical steps. In parallel, it may be desirable to collaborate with a trusted, sophisticated vendor perhaps to effectively collect and use this data in developing a scalable system to decrease surgeon variability.

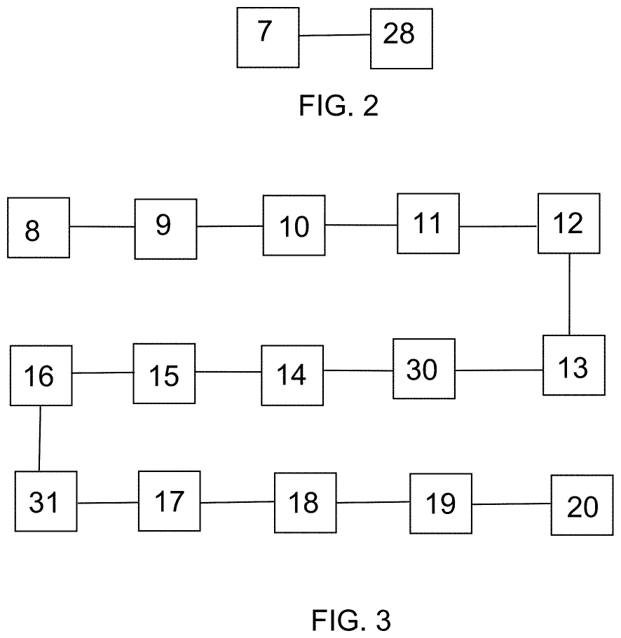

Embodiments of the application may provide pre-surgery analysis (7) of a patient perhaps based on patient data (28) as may be understood in FIG. 2. Patient data (28) may include but is not limited to self-collected patient data, patient imaging data, wearable patient data, patient health records, patient medical records, electronic medical records, electronic health records, magnetic resonance imaging, patient computed tomography scan imaging data, X-ray imaging data, ultrasound imaging data, any combination thereof, and the like. Embodiments of the application can provide AI/ML evaluation of CT, MRI and other medical and radiology imaging. Using AI to evaluate a preoperative MRI or CT can predict optimal operative strategy, use of hardware (which plate, how many screws, etc.), and the like. AI/ML may be used in the evaluation of EHR/EMR, patient acquired data or any other clinical data. As a non-limiting example, wearable data from a patient during pre-operative physical therapy may be used to maximize outcomes such as: physical therapy three times per week for four weeks may be better than two times per week for the same duration, perhaps as evidenced by full range of motion seven days earlier.

As mentioned herein, computer programing may be used to teach a computer and even allow a computer to learn how to identify chapters, use data to plan out an operation, how to identify equipment, instruments, anatomic and pathological structures in recorded images, and the like. In some embodiments, an order of operations may be planned out. This may be based on outcomes from collected data and may provide a recommendation to the order to do things in surgery perhaps considering the most optimal outcomes in pain, healing, and the like. This type of information may be provided selectively perhaps for newer surgeons where advanced surgeons may not need such recommendations. Artificial intelligence (“AI”), machine learning (“ML”), and even computer learning may be utilized in the computer algorithims and the like.

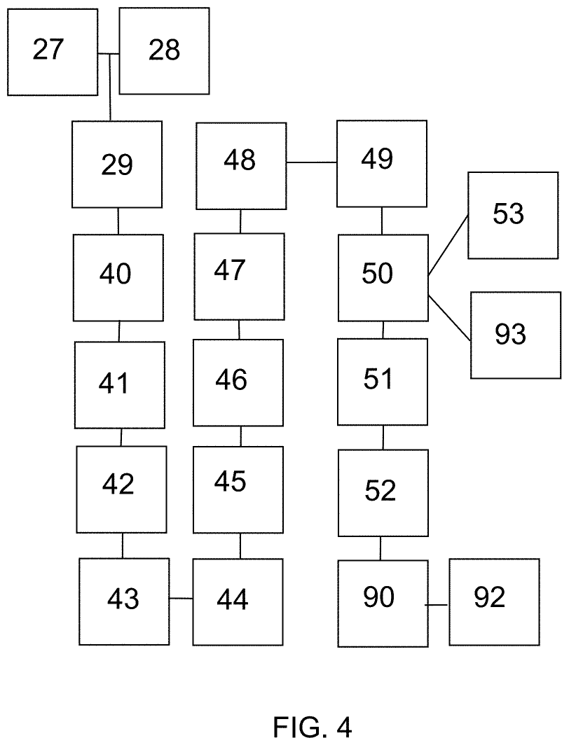

As may be understood from FIG. 3, chapterizing images of a surgery may include automatically accepting a data input (8) to a computer (9) based at least in part on recorded images of a surgery; establishing in a computer a first chapter identification determination model automated computational transform program (10) with starting chapter identification parameters (11); automatically applying a first chapter identification determination model automated computational transform program with starting chapter identification parameters to at least some of data input to automatically create a first chapter identification determination model transform (12); generating a first chapter identification determination model completed output (13) based on first chapter identification determination model transform; automatically varying (30) starting chapter identification parameters for a first chapter identification determination model automated computational transform program to establish a second chapter identification determination model automated computational transform program (14) that differs from a first chapter identification determination model automated computational transform program in the way that it determines chapter identification from data input; automatically applying a second chapter identification determination model automated computational transform program with automatically varied starting chapter identification parameters to at least some of recorded images of a surgery to automatically create a second chapter identification determination model transform (15); generating a different, second chapter identification determination model completed output (16) based on a second chapter identification determination model transform; automatically comparing a first chapter identification determination model completed output with a different, second chapter identification determination model completed output; automatically determining (31) whether a first chapter identification determination model completed output or a different, second chapter identification determination model completed output is likely to provide identification of a new chapter (17); providing a chapter identification indication (18) based on a step of automatically determining whether a first chapter identification determination model completed output or a different, second chapter identification determination model completed output is likely to provide identification of a new chapter; and perhaps even storing (19) automatically improved chapter identification parameters that are determined to identify a new chapter for future use to automatically self-improve (20) chapter identification determination models.

Embodiments may provide creating a baseline (23) for regulatory measures (24) perhaps using collected data (21). Regulatory measures may include but is not limited to Healthcare Effectiveness Data and Information Set, Merit Based Incentive Payments Systems, Medicare Access and Chip Reauthorization Act of 2015, risk sharing models, and the like.

Embodiments of the application may include AI/ML supported Value Based/Capitated Model Clinical Workflows for risk sharing models. AI/ML supported Clinical Compliance Workflows may be used for regulatory requirements such as HEDIS, MIPS/MACRA. Embodiments may provide a collaboration with and even using collecting data associated with new Current Procedural Terminology (“CPT”) codes (25). Embodiments may provide using collected data with

As a non-limiting example, a CPT code for remote kinematic measurement and treatment may be developed as provided below:
0733T: Remote body and limb kinematic measurement-based therapy ordered by a physician or other qualified health care professional; supply and technical support, per 30 days (Dec. 30, 2021 Jul. 1, 2022 CPT® 2023)
0734T: Treatment management services by a physician or other qualified health care professional, per calendar month (Dec. 30, 2021 Jul. 1, 2022 CPT® 2023)
Office-Based Measurement of Mechanomyography and Inertial Measurement Units Code 0778T: represents the measurement and recording of dynamic joint motion and muscle function that includes the incorporation of multiple inertial measurement unit (“IMU”) with concurrent surface mechanomyography (“sMMG”) sensors. Code 0778T is not a remote service and measurements are obtained in the office setting while the patient is physically present. The IMU sensors can contain an accelerometer that measures acceleration and velocity of the body during movement, a gyroscope that measures the positioning, rotation, and orientation of the body during movement, and a magnetometer that measures the strength and direction of the magnetic field to orient the body position during movement relative to the earth's magnetic north field. The sMMG sensors can measure muscle function by quantifying muscle activation and contraction amplitude and duration by recording high-sensitivity volumetric change. A combination of the sensors can be used to dynamically record multi joint motion and muscle function bilaterally and concurrently during functional movement. Data collected from the wireless-enabled IMUs and sMMGs can be uploaded to a secure, Health Insurance Portability and Accountability Act (“HIPAA”)-compliant cloud-based processing platform. The cloud-based application can immediately processes the data and can produce an automated report with digestible chronological data perhaps to assist in serial tracking improvement, decline, or plateau of progress during the episode of care. When 0778T is performed on the same day as another therapy, assessment, or evaluation services, those services may be reported separately and in addition to 0778T.
0778T: Surface mechanomyographs with concurrent application of inertial measurement unit sensors for measurement of multi-joint range of motion, posture, gait, and muscle function (Do not report 0778T in conjunction with 96000, 96004, 98975, 98977, 98980, 98981)

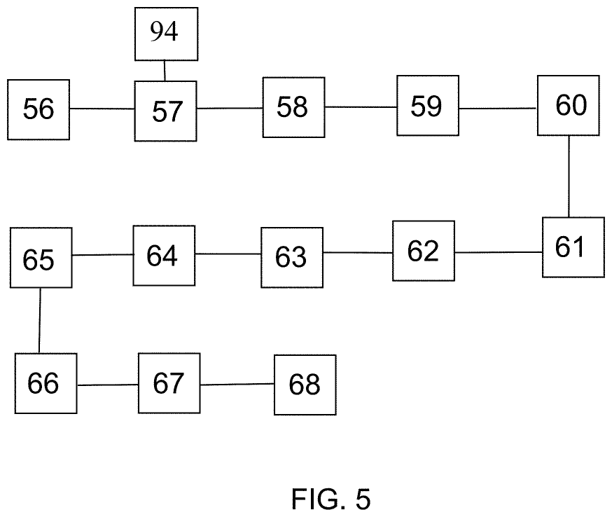

As may be understood from FIG. 4, computer algorithms may be used to prepare an optimal preoperative plan perhaps based on collected data. It may include providing collected data (27) from a plurality of images from different surgeries; providing patient data (28) for a patient; automatically accepting a data input (29) to a computer (40) based at least in part on collected data and patient data; establishing in a computer a first preoperative plan model automated computational transform program (41) with starting preoperative plan parameters (42); automatically applying a first preoperative plan model automated computational transform program with a starting preoperative plan parameters to at least some of data input to automatically create a first preoperative plan model transform (43); generating a first preoperative plan model completed output (44) based on a first preoperative plan model transform; automatically varying the starting preoperative plan parameters for a first preoperative plan model automated computational transform program to establish a second preoperative plan model automated computational transform program (45) that differs from a first preoperative plan model automated computational transform program in the way that it determines preoperative plans (46) from data input; automatically applying a second preoperative plan model automated computational transform program with automatically varied starting preoperative plan parameters to at least some of data input to automatically create a second preoperative plan model transform (47); generating a different, second preoperative plan model completed output based (48) on a second preoperative plan model transform; automatically comparing (49) a first preoperative plan model completed output with a different, second preoperative plan model completed output; automatically determining whether a first preoperative plan model completed output or a different, second preoperative plan model completed output is likely to provide an optimal preoperative plan (50); providing a preoperative plan model identification indication (51) based on a step of automatically determining whether a first preoperative plan model completed output or a different, second preoperative plan model completed output is likely to provide an optimal preoperative plan; and perhaps even storing automatically improved preoperative plan model parameters (52) that are determined to identify optimal preoperative plan for future use to automatically self-improve (90) preoperative plan models. In some embodiments, supplementary data (92) such as cost and even payor claims data or the like may be utilized in self-improvement of preoperative plan models.

An optimal preoperative plan (50) may include data (53) chosen from what instruments to use in the surgery; how long to use instruments in the surgery; what equipment to use in the surgery; techniques to use in the surgery; any combination thereof; and the like. An optimal preoperative plan (50) may include a recommendation (93) including but not limited to surgical selection to maximize patient outcomes and minimize cost; treatment selection to maximize patient outcomes and minimize cost; instrument, implant, or surgical tool selection to be used in a surgery perhaps based on a specific surgical procedure or type of pathology.

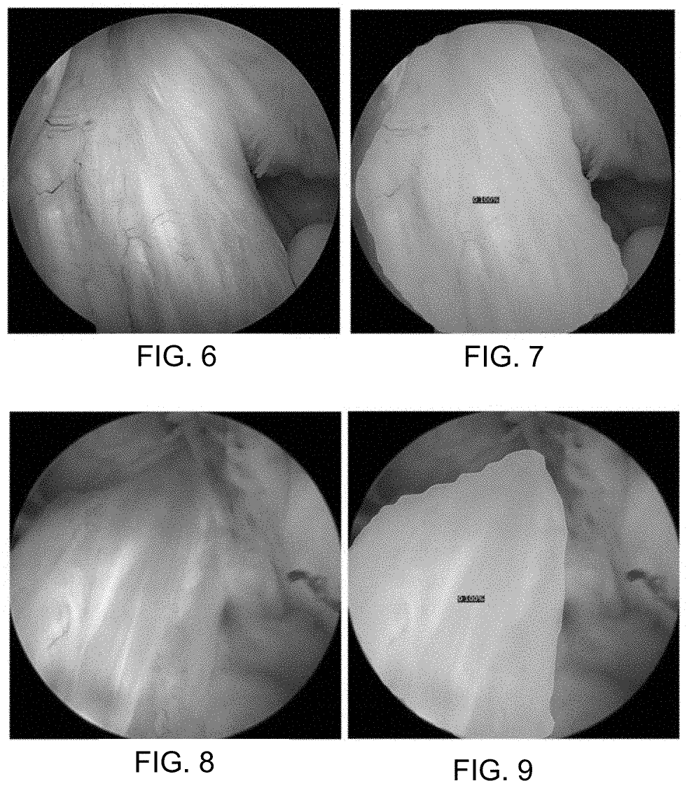

As may be understood from FIG. 5, computer algorithms may be used to identify a surgery characteristics in images. As a non-limiting example, a method may include automatically accepting a data input (56) to a computer (57) based at least in part on recorded images of a surgery; establishing in a computer a first surgery characteristic identification determination model automated computational transform program (58) with starting surgery characteristic identification parameters (59); automatically applying a first surgery characteristic identification determination model automated computational transform program with a starting surgery characteristic identification parameters to at least some of a data input to automatically create a first surgery characteristic identification determination model transform (60); generating a first surgery characteristic identification determination model completed output (61) based on a first surgery characteristic identification determination model transform; automatically varying a starting surgery characteristic identification parameters for a first surgery characteristic identification determination model automated computational transform program to establish a second surgery characteristic identification determination model automated computational transform program (62) that differs from a first surgery characteristic identification determination model automated computational transform program in the way that it determines surgery characteristic identification from data input; automatically applying a second surgery characteristic identification determination model automated computational transform program with an automatically varied starting surgery characteristic identification parameters to at least some of the recorded images of a surgery to automatically create a second surgery characteristic identification determination model transform (63); generating a different, second surgery characteristic identification determination model completed output (64) based on a second surgery characteristic identification determination model transform; automatically comparing a first surgery characteristic identification determination model completed output with a different, second surgery characteristic identification determination model completed output; automatically determining whether a first surgery characteristic identification determination model completed output or a different, second surgery characteristic identification determination model completed output is likely to provide identification of a surgery characteristic (65); providing a surgery characteristic identification indication (66) based on a step of automatically determining whether a first surgery characteristic identification determination model completed output or a different, second surgery characteristic identification determination model completed output is likely to provide an identification of a surgery characteristic; and perhaps even storing (67) automatically improved surgery characteristic identification parameters that are determined to identify surgery characteristic for future use to automatically self-improve (68) surgery characteristic identification determination models. Surgery characteristics (65) may include but is not limited to surgeon's hands, equipment, instruments, anatomic structures, pathological structures, and the like.

In some embodiments, computer algorithms such as AI, ML and CT, and may be used during surgery or in analysis after surgeries to automatically compute (94): automatically determine a type of pathology that is associated with a type of instrument, a type of implant, or a type of surgical intervention based on collected data from other surgeries; automatically associate a case type from surgical common procedural codes (CPT's); automatically determine a collective of an amount of time usage of an instrument or implant per a CPT code, a procedure, or pathology identified; automatically determine a frequency or a type in which an implant is utilized per type of a procedure or pathology encountered; automatically determine a type and an amount of time instrument utilization per procedure coding or pathology encountered; automatically determine implant or instrument variations over time based on collected data; automatically compare a time usage for implants in different surgeries and automatically determine similar pathology among different surgeries; and perhaps even automatically compare instrument use time in different surgeries and automatically determine similar pathology among different surgeries.

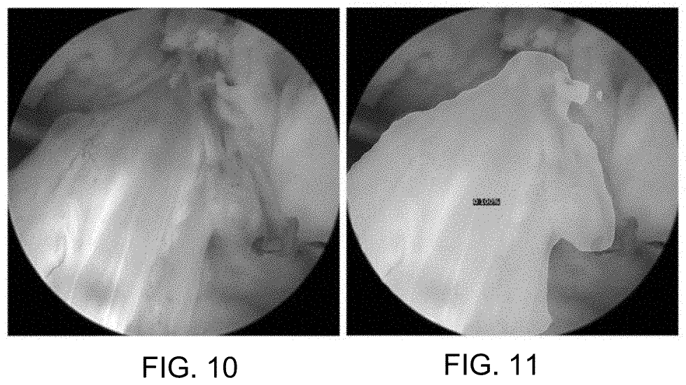

As may be understood from FIG. 6, a representative intraoperative image of the anterior cruciate ligament of the knee is shown which can typically be identified by the surgeon by visual inspection.

As may be understood from FIG. 7, demonstration of an embodiment which correctly identifies the ligament for subsequent use. All anatomic and pathologic structures as well as surgical instruments can have labels such as this.

As may be understood from FIGS. 8 and 10, a representative intraoperative image of the anterior cruciate ligament of the knee is shown which can typically be identified by the surgeon by visual inspection. Note is made of a metallic instrument at the left aspect of the field of view.

As may be understood from FIGS. 9 and 11, demonstration of an embodiment which correctly identifies the ligament with instrumentation manipulating the structure.

As can be easily understood from the foregoing, the basic concepts of the various embodiments of the present invention(s) may be embodied in a variety of ways. It involves both surgery analysis techniques as well as devices to accomplish the appropriate surgery analysis. In this application, the surgery analysis techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “transform” should be understood to encompass disclosure of the act of “transforming”—whether explicitly discussed or not—and, conversely, were there effectively disclosure of the act of “transforming”, such a disclosure should be understood to encompass disclosure of a “transform” and even a “means for transforming.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components, programming, subroutines, logic, or elements that are configured to, or configured and arranged to, provide or even achieve a particular result, use, purpose, situation, function, or operation, or as components that are capable of achieving a particular activity, result, use, purpose, situation, function, or operation. All should be understood as within the scope of this disclosure and written description.

Any regulations, or rules mentioned in this application for patent or patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the information statement filed with the application are hereby appended and hereby incorporated by reference, however, as to each of the above, to the extent that such information or statements incorporated by reference might be considered inconsistent with the patenting of the various embodiments of invention(s) such statements are expressly not to be considered as made by the applicant(s).

Thus, the applicant(s) should be understood to have support to claim and make claims to embodiments including at least: i) each of the surgery analysis devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, it should be understood that in characterizing these and all other aspects of the various embodiments of the invention(s)—whether characterized as a device, a capability, an element, or otherwise, because all of these can be implemented via software, hardware, or even firmware structures as set up for a general purpose computer, a programmed chip or chipset, an ASIC, application specific controller, subroutine, logic, or other known programmable or circuit specific structure—it should be understood that all such aspects are at least defined by structures including, as person of ordinary skill in the art would well recognize: hardware circuitry, firmware, programmed application specific components, and even a general purpose computer programmed to accomplish the identified aspect. For such items implemented by programmable features, the applicant(s) should be understood to have support to claim and make a statement of invention to at least: xv) processes performed with the aid of or on a computer, machine, or computing machine as described throughout the above discussion, xvi) a programmable apparatus as described throughout the above discussion, xvii) a computer readable memory encoded with data to direct a computer comprising means or elements which function as described throughout the above discussion, xviii) a computer, machine, or computing machine configured as herein disclosed and described, xix) individual or combined subroutines, processor logic, and/or programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) a processor executing instructions that act to achieve the steps and activities detailed, xxvi) circuitry configurations (including configurations of transistors, gates, and the like) that act to sequence and/or cause actions as detailed, xxvii) computer readable medium(s) storing instructions to execute the steps and cause activities detailed, xxviii) the related methods disclosed and described, xxix) similar, equivalent, and even implicit variations of each of these systems and methods, xxx) those alternative designs which accomplish each of the functions shown as are disclosed and described, xxxi) those alternative designs and methods which accomplish each of the functions shown as are implicit to accomplish that which is disclosed and described, xxxii) each feature, component, and step shown as separate and independent inventions, and xxxiii) the various combinations of each of the above and of any aspect, all without limiting other aspects in addition.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrases “comprising”, “including”, “containing”, “characterized by” and “having” are used to maintain the “open-end” claims herein, according to traditional claim interpretation including that discussed in MPEP § 2111.03. Thus, unless the context requires otherwise, it should be understood that the terms “comprise” or variations such as “comprises” or “comprising”, “include” or variations such as “includes” or “including”, “contain” or variations such as “contains” and “containing”, “characterized by” or variations such as “characterizing by”, “have” or variations such as “has” or “having”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method for analyzing surgeries comprising the steps of:
recording images of a surgery with a camera, wherein said images comprise a visual element chosen from a surgeon's hands during said surgery, a patient's surgery area, equipment used in said surgery, and instruments used in said surgery;
saving said images of said surgery;
displaying a timestamp in said images;
chapterizing said images of said surgery into different chapters; and
analyzing said recorded images of said surgery.

2. The method as described in claim 1 wherein said images of said surgery comprises moving images and wherein said camera comprises a video camera.

3. The method as described in claim 1 and further comprising a step of pre-surgery analysis of said patient based on patient data chosen from wearable patient data, patient health records, patient medical records, patient magnetic resonance imaging, patient computed tomography scan, and any combination thereof.

4. The method as described in claim 1 wherein said chapters of said recorded images categorize said images of said surgery by categories chosen from when specific equipment is used, when specific instrument is used, and each surgical step.

5. The method as described in claim 4 wherein said chapters of said images include said timestamp to show the timeframe for each chapter.

6. The method as described in claim 4 and further comprising a step of starting a new chapter with said images when a new instrument is used or when an instrument is removed in said surgery.

7. The method as described in claim 1 wherein said step of chapterizing said images of said surgery comprises the steps of:
automatically accepting a data input to a computer based at least in part on said recorded images of said surgery;
establishing in said computer a first chapter identification determination model automated computational transform program with starting chapter identification parameters;
automatically applying said first chapter identification determination model automated computational transform program with said starting chapter identification parameters to at least some of said data input to automatically create a first chapter identification determination model transform;
generating a first chapter identification determination model completed output based on said first chapter identification determination model transform;
automatically varying said starting chapter identification parameters for said first chapter identification determination model automated computational transform program to establish a second chapter identification determination model automated computational transform program that differs from said first chapter identification determination model automated computational transform program in the way that it determines chapter identification from said data input;
automatically applying said second chapter identification determination model automated computational transform program with said automatically varied starting chapter identification parameters to at least some of said recorded images of said surgery to automatically create a second chapter identification determination model transform;
generating a different, second chapter identification determination model completed output based on said second chapter identification determination model transform;
automatically comparing said first chapter identification determination model completed output with said different, second chapter identification determination model completed output;
automatically determining whether said first chapter identification determination model completed output or said different, second chapter identification determination model completed output is likely to provide identification of a new chapter;
providing a chapter identification indication based on said step of automatically determining whether said first chapter identification determination model completed output or said different, second chapter identification determination model completed output is likely to provide said identification of said new chapter; and
storing automatically improved chapter identification parameters that are determined to identify said new chapter for future use to automatically self-improve chapter identification determination models.

8. The method as described in claim 1 wherein said step of analyzing said images of said surgery comprises a step chosen from:
identifying anatomic structures of said patient's surgery area;
identifying pathologic structures of said patient's surgery area;
identifying each equipment or instrument used in said surgery;
identifying equipment or instrument duration of use;
identifying equipment or instrument placement;
identifying equipment or instrument selection;
identifying surgeon technique in said surgery;
comparing said surgery with recorded images of another surgery; and
any combination thereof.

9. The method as described in claim 8 and further comprising a step of collecting data from said step of analyzing said images of said surgery to provide collected data.

10. The method as described in claim 9 and further comprising a step of creating a dynamic surgical scorecard of said surgery from said collected data.

11. The method as described in claim 10 wherein said dynamic surgical scorecard can be created for an agenda chosen from a surgeon, type of surgery, practice of surgeons, type of instrument used, type of instrument used, and type of said surgery area.

12. The method as described in claim 9 and further comprising a step of creating a baseline for regulatory measures using said collected data.

13. The method as described in claim 9 wherein said regulatory measures are chosen from Healthcare Effectiveness Data and Information Set, Merit Based Incentive Payments Systems, Medicare Access and Chip Reauthorization Act of 2015, and risk sharing models.

14. The method as described in claim 9 and further comprising a step of using said collected data with Current Procedural Terminology codes.

15. The method as described in claim 9 and further comprising a step of utilizing said collected data in pre-operation evaluation.

16. The method as described in claim 1 and further comprising the steps of:
providing collected data from a plurality of images from different surgeries;
providing patient data for said patient;
automatically accepting a data input to a computer based at least in part on said collected data and said patient data;
establishing in said computer a first preoperative plan model automated computational transform program with starting preoperative plan parameters;
automatically applying said first preoperative plan model automated computational transform program with said starting preoperative plan parameters to at least some of said data input to automatically create a first preoperative plan model transform;
generating a first preoperative plan model completed output based on said first preoperative plan model transform;
automatically varying said starting preoperative plan parameters for said first preoperative plan model automated computational transform program to establish a second preoperative plan model automated computational transform program that differs from said first preoperative plan model automated computational transform program in the way that it determines preoperative plans from said data input;
automatically applying said second preoperative plan model automated computational transform program with said automatically varied starting preoperative plan parameters to at least some of said data input to automatically create a second preoperative plan model transform;
generating a different, second preoperative plan model completed output based on said second preoperative plan model transform;
automatically comparing said first preoperative plan model completed output with said different, second preoperative plan model completed output;
automatically determining whether said first preoperative plan model completed output or said different, second preoperative plan model completed output is likely to provide an optimal preoperative plan;
providing a preoperative plan model identification indication based on said step of automatically determining whether said first preoperative plan model completed output or said different, second preoperative plan model completed output is likely to provide said optimal preoperative plan; and
storing automatically improved preoperative plan model parameters that are determined to identify said optimal preoperative plan for future use to automatically self-improve preoperative plan models.

17. The method as described in claim 16 wherein said patient data or said collected data is sourced from electronic medical records or electronic health records; and further comprising a step of utilizing said patient data and said collected data to self-improve said preoperative plan models.

18. The method as described in claim 16 wherein said patient data comprises data chosen from self-collected patient data, personal wearable technology data, patient imaging data, magnetic resonance imaging data, computed tomography imaging data, X-ray imaging data, ultrasound imaging data; and further comprising a step of utilizing said patient data to self-improve said preoperative plan models.

19. The method as described in claim 16 and further comprising the steps of:
providing supplementary data chosen from cost and payor claims data; and
utilizing said supplementary data to self-improve said preoperative plan models.

20. The method as described in claim 16 wherein said optimal preoperative plan comprises a recommendation chosen from:
surgical selection to maximize patient outcomes and minimize cost;
treatment selection to maximize patient outcomes and minimize cost; and
instrument, implant, or surgical tool selection to be used in said surgery based on a specific surgical procedure or type of pathology.

21. The method as described in claim 1 and further comprising the steps of:
automatically determine a type of pathology that is associated with a type of instrument, a type of implant, or a type of surgical intervention based on collected data from other surgeries;
automatically associate a case type from surgical common procedural codes (CPT's);
automatically determine a collective of an amount of time usage of an instrument or implant per a CPT code, a procedure, or pathology identified;
automatically determine a frequency or a type in which an implant is utilized per type of a procedure or pathology encountered;
automatically determine a type and an amount of time instrument utilization per procedure coding or pathology encountered;
automatically determine implant or instrument variations over time based on collected data;
automatically compare a time usage for implants in different surgeries and automatically determine similar pathology among different surgeries; and
automatically compare instrument use time in different surgeries and automatically determine similar pathology among different surgeries.

22. The method as described in claim 16 wherein said optimal preoperative plan comprises data chosen from what instruments to use in the surgery; how long to use instruments in the surgery; what equipment to use in the surgery; techniques to use in the surgery; and any combination thereof.

23. The method as described in claim 9 and further comprising a step of providing a real-time analysis of said surgery during said surgery wherein said real-time analysis is based on a comparison of said collected data and data input of said surgery.

24. The method as described in claim 23 wherein said real-time analysis provides a recommendation to said surgeon in said surgery for a surgery step.

25. The method as described in claim 1 and further comprising the steps of:
automatically accepting a data input to a computer based at least in part on said recorded images of said surgery;
establishing in said computer a first surgery characteristic identification determination model automated computational transform program with starting surgery characteristic identification parameters;
automatically applying said first surgery characteristic identification determination model automated computational transform program with said starting surgery characteristic identification parameters to at least some of said data input to automatically create a first surgery characteristic identification determination model transform;
generating a first surgery characteristic identification determination model completed output based on said first surgery characteristic identification determination model transform;
automatically varying said starting surgery characteristic identification parameters for said first surgery characteristic identification determination model automated computational transform program to establish a second surgery characteristic identification determination model automated computational transform program that differs from said first surgery characteristic identification determination model automated computational transform program in the way that it determines surgery characteristic identification from said data input;
automatically applying said second surgery characteristic identification determination model automated computational transform program with said automatically varied starting surgery characteristic identification parameters to at least some of said recorded images of said surgery to automatically create a second surgery characteristic identification determination model transform;
generating a different, second surgery characteristic identification determination model completed output based on said second surgery characteristic identification determination model transform;
automatically comparing said first surgery characteristic identification determination model completed output with said different, second surgery characteristic identification determination model completed output;
automatically determining whether said first surgery characteristic identification determination model completed output or said different, second surgery characteristic identification determination model completed output is likely to provide identification of a surgery characteristic;
providing a surgery characteristic identification indication based on said step of automatically determining whether said first surgery characteristic identification determination model completed output or said different, second surgery characteristic identification determination model completed output is likely to provide said identification of said surgery characteristic; and
storing automatically improved surgery characteristic identification parameters that are determined to identify said surgery characteristic for future use to automatically self-improve surgery characteristic identification determination models.

26. The method as described in claim 25 wherein said surgery characteristic is chosen from surgeon's hands, equipment, instruments, anatomic structures, and pathological structures.
